(19) United States
(12) Patent Application Publication
(10) Pub. No.: US 2013/0316000 A1
(43) Pub. Date: Nov. 28, 2013

(54) COMPOSITIONS AND METHODS FOR TREATMENT OF IRRITABLE BOWEL SYNDROME WITH 5-AMINOSALICYLATE

(71) Applicants: Enoch Bortey, Chapel Hill, NC (US); William Forbes, Raleigh, NC (US)

(72) Inventors: Enoch Bortey, Chapel Hill, NC (US); William Forbes, Raleigh, NC (US)

(21) Appl. No.: 13/832,440

(22) Filed: Mar. 15, 2013

Related U.S. Application Data
(60) Provisional application No. 61/649,268, filed on May 19, 2012.
(60) Provisional application No. 61/682,154, filed on Aug. 10, 2012.

(51) Int. Cl. A61K 31/196

(52) U.S. Cl. CPC A61K 31/196
USPC 424/490; 514/567

(57) ABSTRACT

Embodiments are directed to compositions and related methods for treating gastrointestinal disorders with a granulated mesalamine formulation. In some embodiments, the gastrointestinal disorder being treated is irritable bowel syndrome (IBS). In some embodiments, the gastrointestinal disorder being treated is diarrhea-predominant IBS (d-IBS).

### RELATED APPLICATIONS

This application claims the benefit of U.S. Provisional Application No. 61/649,268 filed May 19, 2012 and U.S. Provisional Application No. 61/682,154, filed Aug. 10, 2012. The entire contents of each of the aforementioned applications are incorporated herein by reference.

### BACKGROUND

Irritable bowel syndrome (IBS) is a common functional disorder of the bowel that has a pronounced effect on quality of life. A defining characteristic of IBS is abdominal discomfort or pain. The Rome III Diagnostic Criteria (a system for diagnosing functional gastrointestinal disorders based on symptoms) for IBS are the accepted current standard for diagnosing IBS in the clinical setting and are consistent with FDA guidance. Table 1 below outlines the criteria for diagnosing and subtyping IBS using Rome III.

Other symptoms that support the diagnosis of IBS include pain; abnormal stool passage (straining, urgency, or feeling of incomplete evacuation); passage of mucus; and bloating or feeling of abdominal distension. Patients can be sub-divided by their underlying bowel habits: (i) diarrhea-predominant IBS, (ii) constipation-predominant IBS, and (ii) constipation alternating with diarrhea (alternating IBS).

The pathophysiology of IBS is poorly understood despite the fact that about a quarter of the population in the UK may exhibit the symptoms, and approximately 15 percent of U.S. adults report symptoms that are consistent with the diagnosis of IBS. It is estimated that only 25 percent of persons with IBS seek medical care. In addition, patients diagnosed with IBS are at increased risk for other, non-gastrointestinal functional disorders such as fibromyalgia and interstitial cystitis.

IBS is the most common diagnosis made by gastroenterologists in the U.S., and accounts for 12 percent of visits to primary care providers. Approximately $8 billion in direct medical costs and $25 billion in indirect costs are spent annually in the U.S. for diagnosing and treating IBS. Thus, IBS accounts for a large proportion of annual healthcare costs in the U.S.

Accordingly, there is a need to provide effective methods of treating IBS in patients in need thereof.

### SUMMARY OF THE INVENTION

Embodiments are directed to compositions and related methods for treating gastrointestinal disorders, e.g., inflammatory gastrointestinal disorders, irritable bowel disease, gastrointestinal motility disorders, functional gastrointestinal disorders, gastroesophageal reflux disease (GERD), Crohn's disease, ulcerative colitis, diverticulitis, inflammatory bowel disease, and gastroparesis, with a granulated mesalamine formulation. In some embodiments, the gastrointestinal disorder being treated is irritable bowel syndrome (IBS). In some embodiments, the gastrointestinal disorder being treated is diarrhea-predominant IBS (d-IBS).

In some embodiments, a method of treating a subject having a gastrointestinal disorder is provided, the method comprising administering to the subject an effective amount of a granulated mesalamine formulation; thereby treating the subject. In some embodiments, the subject has a gastrointestinal disorder selected from the group consisting of: irritable bowel disease, gastrointestinal motility disorders, functional gastrointestinal disorders, gastroesophageal reflux disease (GERD), Crohn's disease, ulcerative colitis (UC), active UC, UC in remission, diverticular disease, inflammatory bowel disease, and gastroparesis. In some embodiments, the subject has irritable bowel syndrome with diarrhea (also known as diarrhea-predominant IBS, or d-IBS).

In some embodiments, the effective amount of granulated mesalamine formulation comprises from between about 0.5 to about 4 g per day. In some embodiments, the effective amount of granulated mesalamine formulation comprises about 750 mg per day. In some embodiments, the effective amount of granulated mesalamine formulation comprises about 1.5 g per day. In some embodiments, the effective amount of granulated mesalamine formulation comprises about 3 g per day.

In some embodiments, the granulated mesalamine formulation is administered as one or more 375 mg dosage units. For example, in dosage amounts totaling about 750 mg per day, the formulation can be administered as two 375 mg dosage units. Similarly, in dosage amounts totaling about 1.5 g or 3 g per day, the formulation can be administered as four and eight 375 mg dosage units, respectively, per day.

In some embodiments, the granulated mesalamine formulation is administered as a single daily dosage. In some embodiments, the granulated mesalamine formulation is administered once daily in the morning.

In some embodiments, the subject maintains remission of the gastrointestinal disease after treatment. In some embodiments, the subject remains relapse free of the gastrointestinal disease after treatment.

In some embodiments, the granulated mesalamine formulation is a delayed and/or extended release formulation. In some embodiments, a “delayed and extended” release formulation comprises formulations that first release mesalamine in the ileum and continue to release mesalamine throughout the terminal ileum and colon. In some embodiments, an “extended release” formulation comprises release throughout the lumen of the colon. In some embodiments, a “delayed release” formulation comprises release at between about pH 5.5 to about pH 7. In some embodiments, a “delayed release” formulation comprises release at about pH 6.

Embodiments are also directed to provision of a locally-acting aminosalicylate (e.g., a granulated mesalamine formulation) for the maintenance of remission of irritable bowel syndrome in a subject, for example, humans. In some embodiments, the subject is an adult human. In some embodiments, the subject is a juvenile or child human. In some embodiments, a granulated mesalamine formulation is administered for the maintenance of remission of irritable bowel syndrome in subjects 18 years of age and older.

In some embodiments, tablets or capsules of the granulated mesalamine formulation are administered once daily (e.g., about 0.75 g/day, about 1.5 g/day, or about 3 g/day) in the morning, afternoon or evening with or without food. In some embodiments, the tablets or capsules of the granulated mesalamine formulation are administered once daily in the morning, with or without food. In some embodiments, the granulated mesalamine formulation is not administered with antacids.

In some embodiments, the granulated mesalamine formulation is administered as extended-release tablets or capsules, each comprising about 0.375 g mesalamine. In some embodiments, the dose for maintenance of remission of irritable bowel syndrome in adult subjects comprises 1.5 g (four granulated mesalamine formulation tablets or capsules) orally once daily in the morning without regard to meals.

In some embodiments, the granulated mesalamine formulation disclosed herein and used in the methods described herein can be formulated to release more (in comparison to Asacol® and other formulations of mesalamine) of the active agent, mesalamine, directly to the therapeutic site of action (e.g., terminal ileum and colon) over a more prolonged period, and to decrease systemic availability relative to unencapsulated mesalamine granules. In some embodiments, the mesalamine is released over approximately 7 hours.

In some embodiments, the granulated mesalamine formulation comprises a hard gelatin capsule shell containing a granulated mesalamine formulation which comprises, for example, an inner polymer matrix mesalamine core that is surrounded by an outer flavor coating, a middle coating, and an inner enteric pH dependent (delayed) release coating. The inner coating can dissolve, for example, at pH>6, but resists dissolution in the stomach, where gastric fluid is pH 1 during fasting and approximately pH 4 during a meal.

In some embodiments, following dissolution of the inner coating, the polymer matrix core of the granulated mesalamine provides a mechanism by which mesalamine, the active therapeutic ingredient, is uniformly and slowly released and distributed in the lumen of the colon. In data disclosed in International Patent Publication No. WO 2010-040113, it was observed that the pellets of the granulated mesalamine formulation have a relatively low rate and extent of systemic absorption, and that 85% to 90% of drug reaches the diseased area. The direct and prolonged targeted release of the active agent from a granulated mesalamine formulation in subjects makes the formulation particularly effective as a once daily (QD) dosage regimen.

In some embodiments, the subject being treated is advised that when being administered granulated mesalamine formulation, renal impairment may occur. In some embodiments, the subject's renal function is assessed at the beginning of treatment. In some embodiments, renal function is assessed before initiating therapy with mesalamine. In some embodiments, the subject's renal function is assessed periodically during therapy. In some embodiments, the subject is advised that the granulated mesalamine formulation should be used with caution in subjects with renal disease. In some embodiments, the blood cell counts are monitored in geriatric subjects being administered the granulated mesalamine formulation. In some embodiments, the subject is advised that the granulated mesalamine formulation contains aspartame.

In some embodiments, the subject is advised that the granulated mesalamine formulation should be used with caution with pre-existing liver disease.

In some embodiments, the subject is advised that there are adverse reactions associated with administration of the granulated mesalamine formulation. In some embodiments, the adverse reactions include, for example, (incidence ≧3%) are headache, diarrhea, upper abdominal pain, nausea, nasopharyngitis, flu or flu-like illness, sinusitis. In some embodiments, the granulated mesalamine formulation comprises extended-release tablets or capsules containing 0.375 g mesalamine. In some embodiments, the granulated mesalamine formulation comprises delayed and extended-release tablets or capsules containing 0.375 g mesalamine.

In some embodiments, the subject is advised that mesalamine has been associated with an acute intolerance syndrome that may be difficult to distinguish from a flare of inflammatory bowel disease. Symptoms include, for example, cramping, acute abdominal pain and bloody diarrhea, sometimes fever, headache, and rash. In some embodiments, if acute intolerance syndrome is suspected, the subject is advised to promptly discontinue treatment with the granulated mesalamine formulation.

In some embodiments, the subject is advised that subjects who have experienced a hypersensitivity reaction to sulfasalazine may have a similar reaction to the granulated mesalamine formulation.

In some embodiments, the subject is advised that based on in vitro studies, granulated mesalamine formulation is not expected to inhibit the metabolism of drugs that are substrates of CYP1A2, CYP2C9, CYP2C19, CYP2D6, or CYP3A4.

In some embodiments, the subject is advised that low concentrations of mesalamine and higher concentrations of its N-acetyl metabolite have been detected in human breast milk.

In some embodiments, the subject is advised that a higher incidence of blood dyscrasias, i.e., neutropenia, pancytopenia, in subjects who were 65 years or older who were taking mesalamine-containing products. In some embodiments, the subject is advised that caution should be taken to closely monitor blood cell counts during mesalamine therapy.

In some embodiments, the subject is advised that reproduction studies with mesalamine have been performed in rats and rabbits and have revealed no evidence of impaired fertility or harm to the fetus due to mesalamine.

In some embodiments, the subject is advised that there is no specific antidote for mesalamine overdose; however, therapy for salicylate toxicity may be beneficial in the event of acute overdosage. This includes prevention of further gastrointestinal tract absorption by emesis and, if necessary, by gastric lavage. Fluid and electrolyte imbalance can be corrected by the administration of appropriate intravenous therapy. Adequate renal function should be maintained.

In some embodiments, the subject and/or the healthcare provider is advised that the granulated mesalamine formulation may be a pH dependent delayed-release product and this factor should be considered when treating a suspected overdose.

In some embodiments, the granulated mesalamine formulation comprises a tablet or capsule comprising a delayed- and extended-release dosage form for oral administration. In some embodiments, each tablet or capsule comprises 0.375 g of mesalamine USP (5-aminosalicylic acid, 5-ASA), an anti-inflammatory drug.

In some embodiments, the subject and/or the healthcare provider is advised not to take granulated mesalamine formulation capsules with antacids, because it could affect the way granulated mesalamine formulation dissolves.

In some embodiments, the subject is advised to contact a health care provider if they experience a worsening of symptoms, because it could be due to a reaction to granulated mesalamine formulation.

As used herein, renal impairment, includes minimal change nephropathy, acute and chronic interstitial nephritis, and, rarely, renal failure.

As used herein, salicylate toxicity symptoms include, for example, hematemesis, tachypnea, hyperpnea, tinnitus, deafness, lethargy, seizures, confusion, or dyspnea. Severe intoxication can lead to electrolyte and blood pH imbalance and potentially to other organ (e.g., renal and liver) involvement.

Embodiments are directed to a method of administering granulated mesalamine as a treatment for a gastrointestinal disorder, the method comprising advising a health care worker and/or a subject that subjects previously prescribed corticosteroids followed by granulated mesalamine have a decreased incidence of adverse events; and administering the granulated mesalamine to the patient in order to treat the gastrointestinal disorder.

Embodiments are also directed to a method of decreasing the incidence of adverse events in the subject having a gastrointestinal disorder after the subject has been treated with corticosteroids, the method comprising administering granulated mesalamine to the subject, thereby decreasing the incidence of adverse events.

Embodiments relate to a method of treating a subject having a gastrointestinal disorder, the method comprising advising a health care worker and/or a subject that subjects having a low mucosal score are most likely remain in remission from the gastrointestinal disorder; and administering the granulated mesalamine to the patient in order to treat the gastrointestinal disorder.

Embodiments also relate to a method of treating a subject having a gastrointestinal disorder, the method comprising determining treatment failure from a non-granulated 5-ASA mesalamine formulation; and responsive to such failure, administering to the subject an effective amount of a granulated mesalamine formulation; thereby treating the subject.

Embodiments can also relate to a method of treating a subject having a gastrointestinal disorder, the method comprising administering to the subject an effective amount of a granulated mesalamine formulation, wherein treatment of the subject with another formulation of mesalamine has failed; thereby treating the subject.

Embodiments are directed to a method of treating a subject having a gastrointestinal disorder, the method comprising administering to the subject an effective amount of a granulated mesalamine formulation orally, once daily. In some embodiments, the subject is administered the formulation once daily in the morning.

In some embodiments, the gastrointestinal disorder is irritable bowel syndrome (IBS). In some embodiments, the gastrointestinal disorder is diarrhea-predominant irritable bowel syndrome (d-IBS). In some embodiments, the adverse events are IBS-related adverse events.

In some embodiments, a method of treating or maintaining remission of irritable bowel syndrome with diarrhea is provided, the method comprising administering from between about 0.75 g and about 3 g of a granulated mesalamine formulation orally to the subject once daily. In some embodiments, about 0.75 g of the granulated mesalamine formulation is administered. In some embodiments, about 1.5 g of the granulated mesalamine formulation is administered. In some embodiments, about 3 g of the granulated mesalamine formulation is administered. In some embodiments, the granulated mesalamine formulation is administered without regard to meals. In some embodiments, the granulated mesalamine formulation is administered with food. In some embodiments, the granulated mesalamine formulation is administered without food. In some embodiments, the granulated mesalamine formulation is not co-administered with antacids. In some embodiments, the granulated mesalamine is administered as one or more dosage units of 0.375 g mesalamine tablets or capsules. For example, in an exemplary embodiment, 1.5 g of granulated mesalamine can be administered in a formulation comprising four capsules. In some embodiments, each tablet or capsule comprises 0.375 g mesalamine. In some embodiments, each tablet or capsule comprises from between about 0.25 g to about 0.45 g mesalamine.

### DETAILED DESCRIPTION

It has surprisingly been demonstrated that administration of granulated mesalamine can improve the symptoms of irritable bowel syndrome in patients, particularly when the symptoms experienced by the patients are more severe. Accordingly, provided herein are granulated mesalamine compositions and methods of using the same for treating, preventing or improving and/or modulating the symptoms of a gastrointestinal disorder, including, for example, inflammatory gastrointestinal disorders, irritable bowel disease, gastrointestinal motility disorders, functional gastrointestinal disorders, gastroesophageal reflux disease (GERD), Crohn's disease, ulcerative colitis, diverticulitis, inflammatory bowel disease, and gastroparesis. In some embodiments, the gastrointestinal disorder being treated is irritable bowel syndrome (IBS). In some embodiments, the gastrointestinal disorder being treated is diarrhea-predominant IBS (d-IBS). “Ameliorate,” “amelioration,” “improvement” or the like refers to, for example, a detectable improvement or a detectable change consistent with improvement that occurs in a subject or in at least a minority of subjects, e.g., in at least about 2%, 5%, 10%, 15%, 20%, 25%, 30%, 40%, 50%, 60%, 70%, 75%, 80%, 85%, 90%, 95%, 98%, 100% or in a range between about any two of these values. Such improvement or change can be observed in treated subjects as compared to subjects not treated with a granulated mesalamine formulation as herein disclosed, where the untreated subjects have, or are subject to developing, the same or similar disease, condition, symptom or the like. Amelioration of a disease, condition, symptom or assay parameter can be determined subjectively or objectively, e.g., self assessment by a subject(s), by a clinician's assessment or by conducting an appropriate assay or measurement, including, e.g., a quality of life assessment, a slowed progression of a disease(s) or condition(s), a reduced severity of a disease(s) or condition(s), or a suitable assay(s) for the level or activity(ies) of a biomolecule(s), cell(s) or by detection of BD episodes in a subject. Amelioration can be transient, prolonged or permanent, or it can be variable at relevant times during or after the granulated mesalamine formulation is administered to a subject or is used in an assay or other method described herein or a cited reference, e.g., within timeframes described infra, or about 1 hour after the administration or use of the granulated mesalamine formulation to about 7 days, 2 weeks, 28 days, or 1, 3, 6, 9 months or more after a subject(s) has received such treatment.

The “modulation” of, e.g., a symptom, level or biological activity of a molecule, or the like, refers, for example, that the symptom or activity, or the like is detectably increased or decreased. Such increase or decrease can be observed in treated subjects as compared to subjects not treated with a granulated mesalamine formulation as disclosed herein, where the untreated subjects have, or are subject to developing, the same or similar disease, condition, symptom or the like. Such increases or decreases can be at least about 2%, 5%, 10%, 15%, 20%, 25%, 30%, 40%, 50%, 60%, 70%, 75%, 80%, 85%, 90%, 95%, 98%, 100%, 150%, 200%, 250%, 300%, 400%, 500%, 1000% or more or within any range between any two of these values. Modulation can be determined subjectively or objectively, e.g., by the subject's self assessment, by a clinician's assessment or by conducting an appropriate assay or measurement, including, e.g., quality of life assessments or suitable assays for the level or activity of molecules within a subject. Modulation can be transient, prolonged or permanent or it can be variable at relevant times during or after the granulated mesalamine formulation is administered to a subject or is used in an assay or other method described herein or a cited reference, e.g., within times described infra, or about 1 hour of the administration or use of the granulated mesalamine formulation to about 2 weeks, 28 days, 3, 6, 9 months or more after a subject(s) has been administered the composition.

The term “modulate” can also refer to increases or decreases in the activity of a cell in response to exposure to a granulated mesalamine formulation as disclosed herein, e.g., the inhibition of proliferation and/or induction of differentiation of at least a sub-population of cells in an animal such that a desired end result is achieved, e.g., a therapeutic result of the granulated mesalamine formulation used for treatment can increase or decrease over the course of a particular treatment.

As used herein, a “subject” to be treated or administered a granulated mesalamine composition as disclosed herein includes organisms which are capable of suffering from a bowel disease, a gastrointestinal disorder or other disorder treatable by a granulated mesalamine formulation as disclosed herein, or who could otherwise benefit from the administration of the formulation, such as human and non-human animals. Preferred human animals include human subjects. The term “non-human animals” of the invention includes all vertebrates, e.g., mammals, e.g., rodents, e.g., mice, and non-mammals, such as non-human primates, e.g., sheep, dog, cow, chickens, amphibians, reptiles, etc. Susceptible to a bowel disease or a gastrointestinal disorder is meant to include a subject at risk of developing a bowel disease (BD) or a gastrointestinal disorder, or a person who is in remission from a BD or a gastrointestinal disorder, or a person who can relapse from a BD or a gastrointestinal disorder, e.g., a subject suffering from immune suppression, a subject that has been exposed to a bacterial infection, physicians, nurses, a subject traveling to remote areas known to harbor bacteria that cause travelers' diarrhea, a family history of BD, an aging person, a person with liver damage, a subject in IBS remission, a subject who has had HE episodes in the past, a person with mind HE, a subject with uncontrollable diarrhea, a subject with dIBS, etc.

The term “administration” or “administering” includes routes of introducing a granulated mesalamine formulation as disclosed herein to a subject to perform its intended function. Examples of routes of administration that can be used include injection (e.g. subcutaneous, intravenous, parenterally, intraperitoneally, intrathecal), oral, inhalation, vaginal, rectal and transdermal. The pharmaceutical preparations can be given by forms suitable for each administration route. For example, the preparations can be administered in tablets or capsule form, by injection, inhalation, eye lotion, eye drops, ointment, suppository, infusion; or topically by lotion or ointment. The injection can be bolus or can be continuous infusion. Oral administration is preferred, and exemplary preparations can be administered in tablets or capsule form or by suppository. Depending on the route of administration, a granulated mesalamine formulation can be coated with or disposed in a selected material to protect it from natural conditions that can detrimentally affect its ability to perform its intended function. The granulated mesalamine formulation can be administered alone, or in conjunction with either another agent or agents as described herein or with a pharmaceutically-acceptable carrier, or both. The granulated mesalamine formulation can be administered prior to the administration of the other agent, simultaneously with the agent, or after the administration of the agent. Furthermore, a granulated mesalamine formulation can be administered in a proform, which is converted into its active metabolite, or more active metabolite in vivo.

“Carriers” as used herein include pharmaceutically acceptable carriers, excipients, or stabilizers which are nontoxic to the cell or mammal being exposed thereto at the dosages and concentrations employed. In some embodiments, a physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN, polyethylene glycol (PEG).

Administration “in combination with” one or more further therapeutic agents includes simultaneous (concurrent) and consecutive administration in any order.

The term “obtaining” as in “obtaining a granulated mesalamine formulation” is intended to include purchasing, synthesizing or otherwise acquiring said granulated mesalamine formulation.

The term “pharmaceutical agent composition” (or agent or drug) as used herein refers to a chemical compound, composition, agent or drug capable of inducing a desired therapeutic effect when properly administered to a patient. It does not necessarily require more than one type of ingredient.

As used herein, “selecting subjects who respond,” “selection of subjects who respond” or the like, include, for example, determining that a subject has responded to treatment based on a decrease of BD or IBS symptoms and/or following label instructions to administer a product (e.g., a granulated mesalamine formulation) for a certain period of time or the like. The determination or selection can be based on the label (e.g., package or package insert) instructions or on the subject's subjective assessment of their symptoms or a healthcare provider's or caretaker's assessment of a subject's symptoms.

As used herein, a “monthly responder” is a subject administered a granulated mesalamine formulation as disclosed herein for treating IBS that has a positive response during at least 2 out of 4 weeks based on daily questions for the weekly responses for relief of at least one of the following: (1) abdominal pain, (2) stool consistency, (3) IBS-related bloating, and (4) IBS symptoms. In some embodiments, a monthly responder has a decrease in weekly average abdominal pain score and a reduction in the number of days per week with at least 1 stool with a consistency of greater than or equal to 6 (per the Bristol stool scale) as defined by the Rome III criteria.

As used herein, a “responder” is a subject who is a monthly responder for an IBS symptom as disclosed herein for at least two months of the treatment period. In some embodiments, a “responder” is a subject who is a monthly responder for at least one of the following for at least two months of the treatment period: (1) abdominal pain, (2) stool consistency, (3) IBS-related bloating, and (4) IBS symptoms. In some embodiments, a “responder” is a subject who is a monthly responder for abdominal pain and stool consistency for at least two months of the treatment period.

Responders can also be identified as a d-IBS subject having one or more of the following: Subjects with moderate bloating and abdominal pain, loose stools and/or bothersome urgency. For example, any one of the following criteria can be used to identify subject that are likely to respond to treatment with a granulated mesalamine formulation as disclosed herein: abdominal pain greater than or equal to, for example, 2, 2.5, 3, or 3.5 on a scale of 0-10 (where a score of 0 corresponds to the absence of pain); bloating greater than, for example, 2, 2.5, 3, or 3.5 on a scale of 0-6 (where a score of 0 corresponds to an absence of bloating); loose stools with an average stool consistency score greater than or equal to 3, 3.5, 4, 4.5 on a scale of 1-7 (where a score of 1 corresponds to stool that is hard to pass); or bothersome urgency for example greater than or equal to 3.0, 3.5, 4.0 or 4.5 days with urgency. In some embodiments, two or more of the above-identified criteria can be used to identify subjects that are likely to respond to treatment with the granulated mesalamine formulation. For example, abdominal pain and bloating; abdominal pain and loose stools, abdominal pain and bothersome urgency; abdominal pain, bloating and loose stools, etc.

Responder can also be defined as: 1)≧30% improvement in abdominal pain, <4 in stool consistency, and ≧1 point decrease in daily IBS symptoms; 2)≧30% improvement in abdominal pain, and ≧50% decrease in number of loose/watery stools within a given week comparing to the baseline; 3)≧30% decrease in mean abdominal pain score from baseline using the worst 3 daily entries in a given week; 4)≧30% decrease in the number of days with urgency within a given week comparing to the baseline; 5)≧30% improvement in the selected worst baseline symptom; or 6) daily responder scores of 0 (not at all) or 1 (hardly) at least 50% of the days in a given week; OR 0 (not at all), 1 (hardly) or 2 (somewhat) 100% of days in a given week in the selected worst baseline symptom.

As used herein, a subject is considered to have a “recurrence” when criteria for a response is absent for at least 3 weeks during a 4 week period. Alternatively, “recurrence” can be defined as a worsening of one or more of stool consistency, abdominal pain or stool consistency and abdominal pain.

The Rome III criteria are the accepted current standard for diagnosing IBS in the clinical setting and are consistent with FDA guidance. Table 1 outlines the criteria for diagnosing and subtyping IBS using Rome III.

TABLE 1
Rome III: IBS Diagnosis and Subtyping

Rome III Criteria
1. Recurrent abdominal pain or discomfort at least 3 days per month in the last 3 months, with
symptom onset at least 6 months prior to diagnosis associated with 2 or more of the following:
improvement with defecation; onset associated with a change in frequency of stool; and/or onset
associated with a change in form (appearance) of stool.
2. Rome III Subtyping
1. IBS with constipation (IBS-C) - hard or lumpy stools^{a} ≧25% and loose (mushy) or watery stools^{b}
<25% of bowel movements, in the absence of use of antidiarrheals or laxatives.
2. IBS with diarrhea (IBS-D) - loose (mushy) or watery stools^{b} ≧25% and hard or lumpy stool^{a} <25%
of bowel movements, in the absence of use of antidiarrheals or laxatives.
3. Mixed IBS (IBS-M) - hard or lumpy stools^{a} ≧25% and loose (mushy) or watery stools^{b} ≧25% of
bowel movements, in the absence of use of antidiarrheals or laxatives.
4. Unsubtyped IBS (IBS-U) - insufficient abnormality of stool consistency to meet criteria for IBS-C, -D
or -M.
References: Ersryd et al., Corazziari et al., and Thompson et al
^{a}Bristol Stool Form Scale 1-2 [separate hard lumps like nuts (difficult to pass) or sausage shaped but lumpy].
^{b}Bristol Stool Form Scale 6-7 (fluffy pieces with ragged edges, a mushy stool or watery, no solid pieces, entirely liquid).

In clinical trials, it was surprisingly shown that granulated mesalamine is efficacious in for the treatment of IBS with diarrhea. It was found that a significantly greater proportion of patients were monthly responders for at least two months in the group to which 1500 mg of granulated mesalamine were administered once daily compared to placebo. In addition, patients who experienced greater than the median abdominal pain responded to treatment with granulated mesalamine at higher rates. Patients who had higher than the median levels of C-reactive protein (CRP) also responded to treatment with granulated mesalamine at higher rates.

Accordingly, provided herein are methods of treating, preventing, or alleviating bowel related and/or gastrointestinal disorders comprising administering to a subject in need thereof a therapeutically effective amount of granulated mesalamine. Bowel related disorders (e.g., bowel diseases) and gastrointestinal disorders include one or more of irritable bowel syndrome (IBS), alternating predominant IBS, diarrhea-predominant Irritable Bowel Syndrome (dIBS), inflammatory bowel disease, Crohn's disease, traveler's diarrhea, ulcerative colitis, enteritis, small intestinal bacterial overgrowth, chronic pancreatitis, pancreatic insufficiency, colitis, diverticular disease, hepatic encephalopathy, abdominal pain associated with IBS, pouchitis, gastroparesis, gastrointestinal motility disorders and/or gastroesophageal reflux disease (GERD).

In some embodiments, a method for treating IBS with diarrhea (or d-IBS) is provided, comprising administration of a therapeutically effective amount of granulated mesalamine to a subject in need thereof, wherein administration of granulated mesalamine reduces IBS-related abdominal pain and discomfort by, for example, at least about 20%, 25%, 30%, 35% or more.

In some embodiments, a method of treating d-IBS is provided, comprising administration of a therapeutically effective amount of granulated mesalamine to a subject in need thereof, wherein administration of granulated mesalamine improves stool consistency, for example, a stool consistency score of <4 (Bristol Stool form scale), and improving the average daily IBS score by at least 1.

In some embodiments, at least 25%, 30%, 35%, 40% or more of subjects administered a therapeutically effective amount of granulated mesalamine to treat IBS have at least a 30% reduction in IBS-related abdominal discomfort, a stool consistency score of <4 (Bristol Stool form scale), and a average daily IBS score that is improved by at least 1.

Endpoints for IBS drug development are set forth below. For IBS with diarrhea, the endpoints use co-primary endpoints that include 2 of the major symptoms, abdominal pain and stool consistency (Table 2). These endpoints are designed to be more symptom-specific than global IBS construct endpoints and to address the common definition of IBS from Rome III as abdominal pain or discomfort that is improved by defecation.

TABLE 2
Endpoints for IBS with Diarrhea

| Co-Primary | | |
|---|---|---|
| endpoint | Entry criteria | Responder Definition |
| Pain Intensity | Pain Intensity | Pain Intensity |
| AND | Weekly average of worst | Decrease in weekly average of worst abdominal pain in |
| Stool | abdominal pain in past 24 hours | past 24 hours score of ≧30% compared with baseline |
| Consistency | score of ≧3.0 in a 0 to 10 point | |
| | score | |
| | Stool Consistency | Stool Consistency |
| | Weekly average ≧Type 6 by the | Weekly average ≦ Type 5 by the Bristol stool score. |
| | Bristol stool score. | |
| | | ‘Classification as a responder involves achieving a |
| | | prespecified improvement in symptoms at least 50 |
| | | percent of the time.’ |

Source: Guidance for Industry. Irritable bowel syndrome: Clinical evaluation of products for treatment. FDA Center for Drug Evaluation and Research (CDER) and Center for Biologics Evaluation and Research (CBER); March 2010.

The endpoints set forth above can be used to determine the efficacy of treatment.

Also provided herein are methods of treating bacterial dysbiosis. Bacterial dysbiosis can be viewed as a quantitative or qualitative imbalance which results in the symptoms of IBS, and not an infection per se. Epidemiologic, physiologic, and clinical evidence has emerged suggesting that dysbiosis of the GI microbiota occurs in the pathogenesis of IBS and may be a target for therapy. The GI microbiota in IBS patients have been shown to have less diversity and stability than in healthy subjects.

Also provided herein are methods for treating a subject having altered microbiota in the gut, thereby treating IBS.

Subjects can be selected for treatment of IBS with, for example, granulated mesalamine based on the presence of one or more biomarkers that are indicative of IBS. For example, stress response biomarkers such as HPA axis; immune activation markers such as cytokines, mucosal lymphocytes, mucosal mast cells or proteases; fecal biomarkers such as calprotectin, human β-defensin, or fecal proteases can be used to select subjects for treatment of IBS.

Additionally, identification of small intestional bacterial overgrowth (SIBO) can be used as a marker for IBS. Techniques to identify SIBO include aspiration and direct culture of jejunal contents, and breath testing, e.g., lactulose hydrogen breath tests and glucose breath tests.

Subjects in need thereof include those who have levels of C-reactive protein (CRP) that are between about 2 mg/L and about 5 mg/L. For example, a subject in need thereof can be one who has a CRP level of about 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9 or 5 mg/L, or any level in between. In some embodiments, a subject in need thereof is one who has a CRP level of at least about 2.2 mg/mL. In some embodiments, a subject in need thereof is one who has a CRP level of at least about 4 mg/mL. CRP levels can be measured by diagnostic tests that are known to those of skill in the art. For example, CRP levels can be measured by obtaining a blood sample from the subject and conducting a high-sensitivity CRP (hs-CRP) test on the sample.

Subjects in need thereof also include subjects having or that are susceptible to bowel disease (BD), are in remission from BD, males and/or older subjects with long duration of disease, as disclosed further below.

As used herein, a therapeutically effective amount means an amount effective, upon single or multiple dose administration to a subject which, when administered to a human or non-human subject, is sufficient to provide a therapeutic benefit such as an amelioration of symptoms, e.g., an amount effective to decrease the symptoms of IBS, or maintenance of remission of IBS. In some embodiments, a therapeutically effective amount includes an amount effective, at dosages and for periods of time necessary, to achieve the desired result, e.g., sufficient to treat or prevent IBS or other mesalamine related disorders in a patient or subject. In some embodiments, a therapeutically effective amount includes one in which any toxic or detrimental effects (e.g., side effects) of a granulated mesalamine formulation are outweighed by the therapeutically beneficial effects.

In some embodiments, a therapeutically effective amount include doses from between about 0.5 g to about 4 g/day or from between about 0.75 g to about 3 g/day, specifically about 0.75 g/day, 1.5 g/day or 3 g/day, of mesalamine formulation. Therapeutically effective amounts and dosage regimens include, for example, administering four tablets, capsules, or granules of the formulation once each day, wherein each tablet, capsule, or granule (e.g., loose or in a sachet) comprises about 375 mg of mesalamine. For example, 1.5 g of a mesalamine in a granulated mesalamine formulation can be administered as four capsules which contain granulated mesalamine formulation granules. Similarly, in dosage amounts totaling about 0.75 g or 3 g per day, the formulation can be administered as two and eight 375 mg dosage units, respectively, per day. In some embodiments, a method of treating a subject with a granulated mesalamine formulation who is in need thereof is provided. Identifying a subject in need of such treatment can be in the judgment of a subject or a health care professional and can be subjective (e.g., opinion) or objective (e.g., measurable by a test or diagnostic method). In some embodiments, a method of identifying subjects in need of treatment with a granulated mesalamine formulation is provided, comprising identifying subjects who have previously had and failed treatment with another mesalamine formulation.

In some embodiments, the granulated mesalamine is administered to a subject from between about 1 week to about 6 weeks in duration, from between about 8 weeks to about 12 weeks in duration, or from between 1 day to about 7 days. The granulated mesalamine can be administered from between about 1 day and about 1 year, or from 1 week to about 24 weeks. The granulated mesalamine can be administered, for example, for the remainder of a subject's life. The granulated mesalamine can be administered intermittently or continuously during the course of treatment. Length of treatment can vary depending on the type and length of disease, and the proper length of treatment can be easily determined by one of skill in the art having the benefit of this disclosure.

For any of the embodiments, granulated mesalamine can be administered, for example, once daily, twice daily, three times daily, or four times daily (or more often as necessary for a particular subject) to a subject. In some embodiments, the methods comprise administering the granulated mesalamine once daily to the subject because it can, for example, minimize the side effects and increase patient compliance. Administration of granulated mesalamine at a frequency of twice or three times daily is also contemplated.

As will be readily apparent to one skilled in the art, the useful in vivo dosage or therapeutically effective amount to be administered and the particular mode of administration can vary depending upon the age, weight and mammalian species treated, the particular compounds employed, and the specific use for which these compounds are employed. The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved. One of skill in the art would be able to determine the proper dose for a subject based on this disclosure.

The amount of the granulated mesalamine formulation which will be effective in the treatment of a particular disorder or condition will depend on the nature of the disorder or condition, and can be determined by standard clinical techniques. The precise dose to be employed in the formulation will also depend on the route of administration, and the seriousness of the disease or disorder, and should be decided according to the judgment of the practitioner and each subject's circumstances. The total daily dosage of a granulated mesalamine formulation can range from about 0.5 g to about 4 g per day. For example, in general, the total daily adult dosage of a granulated mesalamine formulation of the present invention ranges from about 0.75 g to about 3 g, or any whole number or fractional amount in between. A single capsule or tablet can be formulated to contain about 250, 275, 375, 450, 525, 550, 575, 750, 800 or 1000 mg of a granulated mesalamine formulation. In some embodiments, a single capsule or tablet contains about 375 mg of a granulated mesalamine.

In some embodiments, a granulated mesalamine formulation is administered to the subject using a pharmaceutically-acceptable formulation. In some embodiments, the granulated mesalamine formulation is provided as a delayed and/or extended release formulation. For example, a “delayed and extended” release formulation can comprise a formulation that first releases mesalamine in the ileum and continues to release mesalamine throughout the terminal ileum and colon. An “extended release” formulation can, for example, comprise a formulation that releases mesalamine throughout the lumen of the colon. In some embodiments, a “delayed release” formulation comprises one that releases mesalamine at between about pH 5.5 to about pH 7. In some embodiments, a “delayed release” formulation comprises one that releases mesalamine at about pH 6.

In some embodiments, a patient is administered granulated mesalamine as described in U.S. Pat. No. 6,277,412; U.S. Pat. No. 6,551,620 or U.S. Patent Publication No. 2003/0133983, and wherein the granulated mesalamine is advantageously in a capsule dosage form. For example, the granulated mesalamine formulation can be an extended-release capsule, containing, for example 0.375 g of mesalamine. In some embodiments, the granulated mesalamine formulation can be a delayed and an extended-release formulation in capsules, containing, for example 0.375 g of mesalamine.

In some embodiments, the granulated mesalamine formulation is a locally-acting aminosalicylate. In some embodiments, the granulated mesalamine formulation maintains the remission of IBS. The maintenance of remission is, for example, in adults and children. Adult, as used herein, includes, for example, subjects 18 years of age and older. The granulated mesalamine formulation can also be administered to treat active IBS. In some embodiments, the granulated mesalamine formulation is administered until active symptoms are alleviated. In some embodiments, the granulated mesalamine formulation is administered during active disease and is continued to maintain remission.

In some embodiments, the granulated mesalamine formulation can be released directly to the therapeutic site of action (e.g. terminal ileum and/or colon) over a prolonged period of time. For example, the formulation can be released at the therapeutic site of action over a period of between about 2 hours and 18 hours, or between about 4 hours and 12 hours, or between about 6 hours and 8 hours or any amount of time in between. In some embodiments, the formulation can be released over a period of about 7 hours. In some embodiments, the granulated mesalamine formulation comprises four units of a dosage form (e.g., pills, capsules, tablets, sachets, granules) taken once daily. The once daily, can be, for example, in the morning, in the afternoon, in the evening or in the night. In some embodiments, morning comprises, for example, between about 3 AM to about noon. Morning can also be, for example, the time after waking from sleep until noon.

In some embodiments, afternoon comprises, for example, between about noon to about 6 PM. Afternoon can also be, for example, the time from lunch until about 6 pm.

In some embodiments, evening comprises, for example, between about 6 PM to about 3 AM. Evening can also be, for example, the time from dinner starting to sleep.

In some embodiments, night comprises, for example, between about 8 PM to about 4 AM. Night can also be, for example, the time during which the sun has gone down and it is dark outside.

In some embodiments, the granulated mesalamine formulation can be taken or administered without regard to food. For example, it can be taken or administered with or without food.

As used herein, “administered with food” or “taken with food” refers to, for example, any food product, solid or liquid, with caloric content. In some embodiments, the food is a solid food with sufficient bulk and fat content that it is not rapidly dissolved and absorbed in the stomach. For example, the food can be a meal, such as breakfast, lunch or dinner. The dosage of granulated mesalamine can be administered to the subject, for example, between about 60 minutes prior to about 2 hours after eating a meal. For example, the dosage of granulated mesalamine can be administered to the subject between about 1 hour prior to about 1 hour after eating a meal. In some embodiments, the dosage is administered about 5, 10, 15, 20, 25, 30, 35, 40, 45, 50, 55 or 60 minutes prior to eating a meal. In some embodiments, the dosage is administered about 5, 10, 15, 20, 25, 30, 35, 40, 45, 50, 55, 60, 65, 70, 75, 80, 85, 90, 95, 100, 105, 110, 115 or 120 minutes after eating a meal. In some embodiments, the dosage is administered simultaneously with the eating of a meal. In some embodiments, the dosage is administered within 15 minutes of eating a meal.

The terms “without food,” “fasted” and “an empty stomach” refer to, for example, the condition of not having consumed solid food for about 1 hour prior to until about 2 hours after such consumption.

In some embodiments, the granulated mesalamine formulation is not co-administered with antacids. It can be advantageous to not take the granulated mesalamine formulation with antacids because it could affect the way granulated mesalamine formulation dissolves.

In some embodiments, 1.5 g of a granulated mesalamine formulation is administered once daily in the morning.

In some embodiments, 1.5 g of a granulated mesalamine formulation is administered once daily in the morning with or without food. In some embodiments, the granulated mesalamine formulation is administered with food. For example, the granulated mesalamine formulation can be taken during food consumption, or it can be taken on a full stomach. In some embodiments, the granulated mesalamine formulation is administered without food. For example, the granulated mesalamine formulation can be taken before or after food consumption, or it can be taken on an empty stomach.

In some embodiments, 1.5 g of a granulated mesalamine formulation is administered once daily in the morning with or without food and without antacids.

In some embodiments, the granulated mesalamine formulation comprises a hard gelatin capsule shell containing a granulated mesalamine formulation which comprises, for example, an inner polymer matrix mesalamine core that is surrounded by an outer flavor coating, a middle coating, and an inner enteric pH dependent (delayed) release coating. The inner coating can dissolve, for example, at pH>5.5, but resists dissolution in the stomach, where gastric fluid is pH 1 during fasting and approximately pH 4 during a meal.

In some embodiments, following dissolution of the inner coating, the polymer matrix core of the granulated mesalamine provides a mechanism by which mesalamine, the active therapeutic ingredient, is uniformly and slowly released and distributed in the lumen of the colon. As disclosed in International Patent Publication No. WO 2010-040113, it was observed that the pellets of the granulated mesalamine formulation have a relatively low rate and extent of systemic absorption, and that 85% to 90% of drug reaches the diseased area. The direct and prolonged targeted release of the active agent from a granulated mesalamine formulation in subjects makes the formulation particularly effective as a once daily (QD) dosage regimen.

In some embodiments, the granulated mesalamine formulation comprises a tablet or capsule comprising a delayed- and extended-release dosage form for oral administration. In some embodiments, each tablet or capsule comprises 0.375 g of mesalamine USP (5-aminosalicylic acid, 5-ASA), an anti-inflammatory drug.

In some embodiments, granulated mesalamine can be administered in combination with other compounds, including for example, antibiotics, chemotherapeutic agents, anti-inflammatory agents, anti-pyretic agents radiosensitizing agents, radioprotective agents, urologic agents, anti-emetic agents, and/or anti-diarrheal agents. For example, cisplatin, carboplatin, docetaxel, paclitaxel, flurouracil, capecitabine, gemcitabine, irinotecan, topotecan, etoposide, mitomycin, gefitinib, vincristine, vinblastine, doxorubicin, cyclophosphamide, celecoxib, rofecoxib, valdecoxib, ibuprofen, naproxen, ketoprofen, dexamethasone, prednisone, prednisolone, hydrocortisone, acetaminophen, misonidazole, amifostine, tamsulosin, phenazopyridine, ondansetron, granisetron, alosetron, palonosetron, promethazine, prochlorperazine, trimethobenzamide, aprepitant, diphenoxylate with atropine, and/or loperamide.

Embodiments are directed to articles of manufacture that comprise, for example, a container holding a pharmaceutical composition suitable for oral administration of granulated mesalamine in combination with printed labeling instructions providing a discussion of when a particular dosage form should be administered if the patient has been diagnosed with IBS. The dosage can be modified for administration to a subject suffering from IBS, or include labeling for administration to a subject suffering from IBS. Exemplary dosage forms and administration protocols are described infra. The composition can be contained in any suitable container capable of holding and dispensing the dosage form and which will not significantly interact with the composition and will further be in physical relation with the appropriate labeling. The labeling instructions can be consistent with the methods of treatment as described hereinbefore. The labeling can be associated with the container by any means that maintain a physical proximity of the two. By way of non-limiting example, they can both be contained in a packaging material such as a box or plastic shrink wrap or can be associated with the instructions being bonded to the container such as with glue that does not obscure the labeling instructions or other bonding or holding means.

Embodiments are also directed to an article of manufacture that comprises a container containing a pharmaceutical composition comprising granulated mesalamine wherein the container holds granulated mesalamine compositions in unit dosage form and is associated with printed labeling instructions advising that administration of the granulated mesalamine can alleviate symptoms of IBS with diarrhea.

Packaged compositions are also provided, and can comprise a therapeutically effective amount of granulated mesalamine capsules. Kits are also provided herein, for example, kits for treating gastrointestinal diseases in a subject. The kits can contain, for example, granulated mesalamine capsules and instructions for use when treating a subject who has been diagnosed with IBS. The instructions for use can contain prescribing information, dosage information, storage information, and the like.

Mesalamine formulations are described in U.S. Pat. No. 6,277,412; U.S. Pat. No. 6,551,620 and U.S. Patent Publication No. 2003/0133983 to Dr. FaIk Pharma GmbH. The entire contents of U.S. Pat. Nos. 6,277,412 and 6,551,620 and of U.S. Patent Publication No. 2003/0133983 are expressly incorporated by reference herein.

Formulations of granulated mesalamine useful in the methods disclosed herein comprise, for example, granulated mesalamine with a pH dependent coating that dissolves at pH 6 or greater, reached in the terminal ileum and colon, and a polymer matrix core which distributes the mesalamine slowly and uniformly throughout the lumen of the terminal ileum and colon. Thus, the formulation is, for example, delayed because it does not release the mesalamine until the terminal ileum, and it is also extended release because it continuously releases mesalamine throughout the terminal ileum and the colon. This release profile is particularly advantageous to treat bowel diseases such as IBS.

In some embodiments, the encapsulated granulated mesalamine formulation are encapsulated in capsules, for example, hard gelatin, size “00” capsule shells.

In some embodiments, each granulated mesalamine formulation capsule contains, for example, granules composed of mesalamine in a polymer matrix with an enteric coating that dissolves at pH 6 and above. Inactive ingredients of granulated mesalamine formulation capsules can include one or more of, for example, colloidal silicon dioxide, magnesium stearate, microcrystalline cellulose, simethicone emulsion ethylacrylate/methylmethacrylate copolymer nonoxynol 100 dispersion (Eudragit NE40D), hypromellose, polymethacrylic acid:methylmethacrylic acid copolymer in a 1:1 ratio (Eudragit L100), talc, titanium dioxide, triethyl citrate, aspartame, anhydrous citric acid, povidone (K25), vanilla flavoring agent, and edible black ink.

In some embodiments, the composition of granulated mesalamine formulations can be contained in a hard-shell capsule, which is a delayed and extended release dosage form for oral administration. Each capsule can contain, for example, 0.375 g of mesalamine USP (5-amino salicylic acid, 5-ASA), an anti-inflammatory drug. The structural formula of mesalamine is:

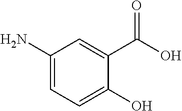 [SMILES: Nc1ccc(O)c(C(=O)O)c1]

Molecular Weight: 153.135

Molecular Formula: C7H7NO3

As discussed below in the Examples, in a randomized, double-blind, placebo-controlled, phase II study, higher numbers of granulated mesalamine (GM)-treated patients than placebo-treated patients reported positive responses in alleviation of IBS symptoms. This analysis investigated the efficacy of various daily doses of mesalamine granules to provide adequate relief from symptoms of IBS with diarrhea. Patients who met the Rome III diagnostic criteria for IBS were randomized to receive 750 mg or 1.5 g granulated mesalamine in capsules, or a placebo, once daily for 12 weeks. The primary efficacy endpoint was the proportion of patients who were monthly responders in both IBS-related abdominal pain and stool consistency for at least two months during the entire three month period of the study. A monthly responder is a subject who achieves weekly adequate relief for at least two weeks of the month, and weekly adequate relief was considered achieved if score thresholds defined for the study were met.

Demographics and baseline characteristics were similar between groups (n=47 750 mg GM, n-51 1500 mg GM, n=50 placebo). The results of the study suggested better response rates for GM dosed at 1.5 g once daily when baseline abdominal pain is greater than the median, for example, when patients had an initial baseline level of C-reactive protein (CRP) of greater than the median.

### EXAMPLES

It will be appreciated that the invention should not be construed to be limited to the example, which is now described; rather, the invention is construed to include any and all applications provided herein and all equivalent variations within the skill of the ordinary artisan.

### Example 1

### Efficacy of Granulated Mesalamine for Treatment of Diarrhea-Predominant Irritable Bowel Syndrome (dIBS)

The study was a 12-week, randomized, placebo-controlled, double-blind multicenter study to assess the efficacy and safety of mesalamine granules for treatment of irritable bowel syndrome with diarrhea. One hundred and forty-eight (148) subjects, meeting the definition of d-IBS as indicated by the Rome III Criteria for the diagnosis of IBS were randomized into three groups: placebo, 750 mg once daily (QD) and 1500 mg once daily (QD). The study consisted of a screening phase, followed by a treatment phase of 12 weeks during which the study drug (mesalamine granules or placebo) was administered to subjects, followed by a treatment phase, during which an end of study visit or phone call at 5±2 days post-end of treatment was made. The total study duration, including the screening phase, was approximately 16 weeks.

There were few serious adverse events. Adverse event rates were similar across all dose groups as shown in Table 3.

TABLE 3
Summary of Adverse Events

| | Placebo | MG 750 mg | MG 1500 mg | Total |
|---|---|---|---|---|
| | [N = 50] | [N = 47] | [N = 51] | [N = 148] |
| Category | n (%) | n (%) | n (%) | n (%) |
| Subjects with Any TEAE | 25 (50.0%) | 26 (55.3%) | 28 (54.9%) | 79 (53.4%) |
| TEAE Intensity [1] | | | | |
| Severe | 1 (2.0%) | 2 (4.3%) | 3 (5.9%) | 6 (4.1%) |
| Moderate | 12 (24.0%) | 12 (25.5%) | 14 (27.5%) | 38 (25.7%) |
| Mild | 12 (24.0%) | 12 (25.5%) | 11 (21.6%) | 35 (23.6%) |
| Subjects Reporting TEAEs Related to | 8 (16.0%) | 9 (19.1%) | 11 (21.6%) | 28 (18.9%) |
| Study Drug | | | | |
| Subjects with Treatment-Emergent | 0 | 1 (2.1%) | 0 | 1 |
| Serious Adverse Events | | | | |
| Subjects Discontinued Study Drug Due to | 2 (4.0%) | 2 (4.3%) | 0 | 4 (2.7%) |
| TEAE | | | | |
| Deaths | 0 | 0 | 0 | 0 |

Note:
TEAE = treatment emergent adverse event. A treatment-emergent AE (TEAE) is defined as any event with a start date occurring on or after treatment Day 1 and before last dose date plus 5 days. Death is considered as treatment-emergent if it occurs on or after treatment Day 1 and before last dose date plus 30 days.

The outcome was measured in terms of the number of months that subjects are monthly responders in both IBS-related abdominal pain and stool consistency during the entire three months. A weekly responder in abdominal pain is defined as a ≧30% improvement from baseline in the weekly average abdominal pain score on a 10-point scale (0=no pain-10=worst possible pain). A weekly responder in stool consistency is defined as ≧50% reduction in the number of days in a week with stool consistency of Type 6 or 7 compared with baseline using the Bristol Stool Scale. Monthly responders are subjects who are weekly responders in both abdominal pain and stool consistency for at least two out of four weeks.

Demographics and baseline characteristics were similar between groups. As indicated in Table 4, it was observed that a significantly greater proportion of patients were monthly responders for the entire three month treatment period in the group to which 1500 mg of granulated mesalamine were administered once daily compared to the placebo group. A patient receiving 1500 mg of granulated mesalamine once daily is about twice as likely to be a monthly responder relative to a patient receiving placebo.

TABLE 4
Efficacy Analysis: Overall Responder in IBS-Related
Abdominal Pain and Stool Consistency During the Entire
Three-Month Treatment Period by Month

| | Placebo | MG 750 mg | MG 1500 mg |
|---|---|---|---|
| | [N = 50] | [N = 47] | [N = 51] |
| Efficacy Endpoints | n (%) | n (%) | n (%) |
| 0 month | 28 | 27 | 18 |
| 1 month | 8 | 5 | 9 |
| 2 month | 6 | 5 | 13 |
| 3 month | 8 | 10 | 11 |

In a statistical analysis for the number of months that subjects are monthly responders in both IBS-related abdominal pain and stool consistency during the three-month treatment period [1500 mg vs. Placebo], it was found that statistical significant treatment resulted from treatment with 1500 mg of mesalamine granules (P-Value of 0.0327; an odds ratio (OR) of 2.232 and a 95% Confidence Interval of 1.068 to 4.664).

An analysis of the percentage of patients who were monthly responders in both abdominal pain and stool consistency for at least two months during the three-month treatment period was also compared between groups. A monthly responder is a patient who is a weekly responder in both abdominal pain and stool consistency for at least two out of four weeks.

As indicated in Table 5, it was observed that a significantly greater proportion of patients were monthly responders for both abdominal pain and stool consistency for at least two months in the group to which 1500 mg of granulated mesalamine were administered once daily compared to the placebo group (47.1% vs. 28%, p=0.0432). Odds ratio calculations for the proportion of subjects who are monthly responders for both abdominal pain and stool consistency for at least two months of the three-month treatment period are provided in Table 8 (1500 mg treatment group relative to the 750 mg treatment group) and in Table 9 (750 mg treatment group relative to the placebo group). As indicated, a patient receiving 1500 mg of granulated mesalamine once daily is about twice as likely to be a monthly responder relative to a patient receiving 750 mg of granulated mesalamine.

1500 mg of mesalamine granules administered once daily provided a statistically significant improvement in abdominal pain and stool consistency in patients with IBS-D.

TABLE 5
Efficacy Analysis: Overall Responder in IBS-Related
Abdominal Pain and Stool Consistency by Treatment Group

| | Placebo | MG 750 mg | MG 1500 mg |
|---|---|---|---|
| | [N = 50] | [N = 47] | [N = 51] |
| Efficacy Endpoints | n (%) | n (%) | n (%) |
| IBS-Related Abdominal | | | |
| Pain and Stool | | | |
| Consistency [2] | | | |
| Responder | 14 (28.0%) | 15 (31.9%) | 24 (47.1%) |
| p-value [1] | | 0.6059 | 0.0432 |
| IBS-Related Abdominal | | | |
| Pain [3] | | | |
| Responder | 26 (52.0%) | 22 (46.8%) | 29 (56.9%) |
| p-value [1] | | 0.7028 | 0.5803 |
| Stool Consistency [4] | | | |
| Responder | 20 (40.0%) | 23 (48.9%) | 31 (60.8%) |
| p-value [1] | | 0.3393 | 0.0333 |

[2] Weekly adequate relief in IBS-related abdominal pain and stool consistency is achieved when a subject achieves both abdominal pain relief and stool consistency relief for a given week.
[3] Weekly adequate relief in abdominal pain is achieved when a subject has a 30% or greater improvement from baseline in the weekly average abdominal pain score.
[4] Weekly adequate relief in stool consistency is achieved when a subject has at least 50% reduction in the number of days in a week with stool consistency of type 6 or 7 on a scale of 1-7. (1 = Separate hard lumps that are hard to pass, 7 = watery stool with no solid pieces).

In a statistical analysis for the proportion of subjects that are monthly responders in both abdominal pain and stool consistency for at least two months during the three-month treatment period [1500 mg vs. 750 mg], it was found that statistical significant treatment resulted from treatment with 1500 mg of mesalamine granules.

In addition, for all efficacy outcomes, a better response rate for the MG 1500 mg treatment group was observed when baseline abdominal pain is greater than the median. Tables 6 and 7 below provide subgroup analysis that supports this observation.

TABLE 6
Overall Responder in IBS-Related Abdominal Pain and Stool
Consistency by Treatment Group and Baseline Abdominal Pain
Baseline Abdominal Pain: >5.60 (Med)

| | Placebo | MG 750 mg | MG 1500 mg |
|---|---|---|---|
| | [N = 30] | [N = 18] | [N = 26] |
| Efficacy Endpoints | n (%) | n (%) | n (%) |
| IBS-Related Abdominal | | | |
| Pain and Stool Consistency | | | |
| [2] | | | |
| Responder | 8 (26.7%) | 8 (44.4%) | 16 (61.5%) |
| p-value [1] | | 0.2099 | 0.0102 |
| IBS-Related Abdominal | | | |
| Pain [3] | | | |
| Responder | 15 (50.0%) | 10 (55.6%) | 17 (65.4%) |
| p-value [1] | | 0.7093 | 0.2482 |
| Stool Consistency [4] | | | |
| Responder | 13 (43.3%) | 10 (55.6%) | 17 (65.4%) |
| p-value [1] | | 0.4133 | 0.1020 |

[2] Weekly adequate relief in IBS-related abdominal pain and stool consistency is achieved when a subject achieves both abdominal pain relief and stool consistency relief for a given week.
[3] Weekly adequate relief in abdominal pain is achieved when a subject has a 30% or greater improvement from baseline in the weekly average abdominal pain score.
[4] Weekly adequate relief in stool consistency is achieved when a subject has at least 50% reduction in the number of days in a week with stool consistency of type 6 or 7 on a scale of 1-7. (1 = Separate hard lumps that are hard to pass, 7 = watery stool with no solid pieces).

TABLE 7
Overall Responder in IBS-Related Abdominal Pain,
Stool Consistency and daily IBS Symptoms by
Treatment Group and Baseline Abdominal Pain
Baseline Abdominal Pain: >5.60 (Med)

| | Placebo | MG 750 mg | MG 1500 mg |
|---|---|---|---|
| | [N = 30] | [N = 18] | [N = 26] |
| Efficacy Endpoints | n (%) | n (%) | n (%) |
| IBS-Related Abdominal | | | |
| Pain [3], Stool Consistency | | | |
| [4] and Daily IBS | | | |
| Symptoms [5] [2] | | | |
| Responder | 7 (23.3%) | 8 (44.4%) | 14 (53.8%) |
| p-value [1] | | 0.1318 | 0.0214 |
| Daily IBS Symptoms Relief | | | |
| [5] | | | |
| Responder | 12 (40.0%) | 12 (66.7%) | 17 (65.4%) |
| p-value [1] | | 0.0781 | 0.0609 |

[2] Weekly success in abdominal pain, stool consistency and daily IBS symptoms is achieved when a subject has relief in abdominal pain, stool consistency and daily IBS symptoms for a given week.
[3] Weekly adequate relief in abdominal pain is achieved when a subject has a 30% or greater improvement from baseline in the weekly average abdominal pain score.
[4] Weekly adequate relief in stool consistency is achieved when a subject has at least 50% reduction in the number of days in a week with stool consistency of type 6 or 7 on a scale of 1-7. (1 = Separate hard lumps that are hard to pass, 7 = watery stool with no solid pieces).
[5] Weekly relief in daily IBS symptoms is achieved when a subject has at least one (1) point decrease in the average daily IBS symptoms score compared with baseline.

In addition, for all efficacy outcomes, a better response rate for the MG 1500 mg treatment group was observed when the baseline level of C-reactive protein (CRP) found in the subject was greater than about 2.2 mg/L. Tables 8-10 below provide subgroup analysis that supports this observation.

TABLE 8
Overall Responder in IBS-Related Abdominal Pain and Stool
Consistency Symptoms by Treatment Group and Baseline CRP
C Reactive Protein: >=2.24 mg/L

| | Placebo | MG 750 mg | MG 1500 mg |
|---|---|---|---|
| | [N = 24] | [N = 23] | [N = 26] |
| Efficacy Endpoints | n (%) | n (%) | n (%) |
| IBS-Related Abdominal | | | |
| Pain and Stool Consistency | | | |
| [2] | | | |
| Responder | 6 (25.0%) | 10 (43.5%) | 17 (65.4%) |
| p-value [1] | | 0.1856 | 0.0056 |
| IBS-Related Abdominal | | | |
| Pain[3] | | | |
| Responder | 11 (45.8%) | 13 (56.5%) | 18 (69.2%) |
| p-value [1] | | 0.4646 | 0.0976 |
| Stool Consistency [4] | | | |
| Responder | 7 (29.2%) | 15 (65.2%) | 19 (73.1%) |
| p-value [1] | | 0.0156 | 0.0028 |

[2] Weekly adequate relief in IBS-related abdominal pain and stool consistency is achieved when a subject achieves both abdominal pain relief and stool consistency relief for a given week.
[3] Weekly adequate relief in abdominal pain is achieved when a subject has 30% or greater improvement from baseline in the weekly average abdominal pain score.
[4] Weekly adequate relief in stool consistency is achieved when a subject has at least 50% reduction in the number of days in a week with stool consistency of type 6 or 7 on a scale of 1-7. (1 = Separate hard lumps that are hard to pass, 7 = watery stool with no solid pieces).

TABLE 9
Overall Responder in IBS-Related Abdominal Pain,
Stool Consistency and daily IBS Symptoms by
Treatment Group and Baseline CRP
C Reactive Protein: >=2.24 mg/L

| | Placebo | MG 750 mg | MG 1500 mg |
|---|---|---|---|
| | [N = 24] | [N = 23] | [N = 26] |
| Efficacy Endpoints | n (%) | n (%) | n (%) |
| IBS-Related Abdominal | | | |
| Pain [3], Stool | | | |
| Consistency [4] and | | | |
| Daily IBS Symptoms | | | |
| [5] [2] | | | |
| Responder | 6 (25.0%) | 10 (43.5%) | 15 (57.7%) |
| p-value [1] | | 0.1856 | 0.0223 |
| Daily IBS Symptoms Relief | | | |
| [5] | | | |
| Responder | 9 (37.5%) | 14 (60.9%) | 19 (73.1%) |
| p-value [1] | | 0.1125 | 0.0135 |

[2] Weekly success in abdominal pain, stool consistency and daily IBS symptoms is achieved when a subject has relief in abdominal pain, stool consistency and daily IBS symptoms for a given week.
[3] Weekly adequate relief in abdominal pain is achieved when a subject has 30% or greater improvement from baseline in the weekly average abdominal pain score.
[4] Weekly adequate relief in stool consistency is achieved when a subject has at least 50% reduction in the number of days in a week with stool consistency of type 6 or 7 on a scale of 1-7. (1 = Separate hard lumps that are hard to pass, 7 = watery stool with no solid pieces).
[5] Weekly relief in daily IBS symptoms is achieved when a subject has at least 1 point decrease in the average daily IBS symptoms score compared with baseline.

TABLE 10
Overall Responder in Global IBS Symptoms and IBS-
Related Bloating by Treatment Group and Baseline CRP
C Reactive Protein: >=2.24 mg/L

| | Placebo | MG 750 mg | MG 1500 mg |
|---|---|---|---|
| | [N = 24] | [N = 23] | [N = 26] |
| Efficacy Endpoints | n (%) | n (%) | n (%) |
| Global IBS Symptoms | | | |
| (Daily Data) [2] | | | |
| Responder | 2 (8.3%) | 5 (21.7%) | 8 (30.8%) |
| p-value [1] | | 0.2120 | 0.0625 |
| Global IBS Symptoms | | | |
| (Weekly Data) [3] | | | |
| Responder | 5 (20.8%) | 11 (47.8%) | 16 (61.5%) |
| p-value[1] | | 0.0561 | 0.0051 |
| IBS-Related Bloating [2] | | | |
| Responder | 1 (4.2%) | 4 (17.4%) | 7 (26.9%) |
| p-value [1] | | 0.1740 | 0.0549 |

[2] Weekly adequate relief in IBS-related bloating/IBS symptoms (daily reported) is achieved when a subject rates his/her daily symptoms as either: 0 (not at all) or 1 (hardly) at least 50% of the days in a given week; OR 0 (not at all), 1 (hardly) or 2 (somewhat) 100% of the days in a given week.
[3] Weekly adequate relief of IBS symptoms (weekly reported) is defined as a response of “yes” to the weekly IBS symptoms question.

For all efficacy outcomes, a better response rate for the MG 1500 mg treatment group was also observed when the baseline level of C-reactive protein (CRP) found in the subject was greater than about 4 mg/L. Table 11 below provides subgroup analysis that supports this observation.

TABLE 11
Overall Responder in IBS-Related Abdominal Pain and
Stool Consistency Symptoms by Treatment Group and
Baseline CRP (Worst)
C Reactive Protein: >=4.11 mg/L

| | Placebo | MG 750 mg | MG 1500 mg |
|---|---|---|---|
| | [N = 13] | [N = 12] | [N = 17] |
| Efficacy Endpoints | n (%) | n (%) | n (%) |
| IBS-Related Abdominal Pain | | | |
| and Stool Consistency [2] | | | |
| Responder | 4 (30.8%) | 6 (50.0%) | 11 (64.7%) |
| p-value [1] | | 0.3305 | 0.0716 |
| IBS-Related Abdominal Pain | | | |
| [3] | | | |
| Responder | 6 (46.2%) | 6 (50.0%) | 12 (70.6%) |
| p-value [1] | | 0.8476 | 0.1812 |
| Stool Consistency [4] | | | |
| Responder | 5 (38.5%) | 7 (58.3%) | 13 (76.5%) |
| p-value [1] | | 0.3237 | 0.0412 |

[2] Weekly adequate relief in IBS-related abdominal pain and stool consistency is achieved when a subject achieves both abdominal pain relief and stool consistency relief for a given week.
[3] Weekly adequate relief in abdominal pain is achieved when a subject has 30% or greater improvement from baseline in the weekly average abdominal pain score.
[4] Weekly adequate relief in stool consistency is achieved when a subject has at least 50% reduction in the number of days in a week with stool consistency of type 6 or 7 on a scale of 1-7. (1 = Separate hard lumps that are hard to pass, 7 = watery stool with no solid pieces).

### INCORPORATION BY REFERENCE

The contents of all references, patents, pending patent applications and published patents, cited throughout this application are hereby expressly incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method of treating a gastrointestinal disorder, comprising administering granulated mesalamine to a subject, wherein the subject has a C-reactive protein (CRP) level of at least 2.2 mg/L.

2. A method of treating a gastrointestinal disorder, comprising administering granulated mesalamine to a subject, wherein the subject has a C-reactive protein (CRP) level of at least 4 mg/L.

3. A method of treating a gastrointestinal disorder, comprising administering granulated mesalamine to a subject, wherein the subject has a C-reactive protein (CRP) level of between about 2 mg/L and about 5 mg/L.

4. The method of any one of claims 1-3, further comprising determining the level of CRP in said subject.

5. The method of claim 4, wherein the level of CRP in said subject is determined by a high-sensitivity CRP (hs-CRP) test.

6. The method of any one of claims 1-3, further comprising obtaining a blood sample from said subject and determining the level of CRP in the blood sample.

7. A method of treating a gastrointestinal disorder in a subject, comprising:
(a) obtaining a sample from said subject;
(b) determining the level of C-reactive protein (CRP) in said sample; and
(c) administering a therapeutically effective amount of granulated mesalamine to the subject if the determined level of CRP is between about 2 mg/L and 5 mg/L,
wherein administration of granulated mesalamine results in treatment of the gastrointestinal disorder.

8. A method of treating a gastrointestinal disorder in a subject, comprising:
(a) obtaining a sample from said subject;
(b) determining the level of C-reactive protein (CRP) in said sample; and
(c) administering granulated mesalamine to the subject if the determined level of CRP is at least about 2.2 mg/L,
wherein administration of granulated mesalamine results in treatment of the gastrointestinal disorder.

9. A method of treating a gastrointestinal disorder in a subject, comprising:
(a) obtaining a sample from said subject;
(b) determining the level of C-reactive protein (CRP) in said sample; and
(c) administering granulated mesalamine to the subject if the determined level of CRP is at least about 4 mg/L,
wherein administration of granulated mesalamine results in treatment of the gastrointestinal disorder.

10. A method of improving the symptoms of a gastrointestinal disorder in a subject, comprising:
(a) obtaining a sample from said subject;
(b) determining the level of C-reactive protein (CRP) in said sample; and
(c) administering granulated mesalamine to the subject if the determined level of CRP is between about 2 mg/L and 5 mg/L,
wherein administration of granulated mesalamine results in the improvement of symptoms of the gastrointestinal disorder.

11. A method of improving the symptoms of a gastrointestinal disorder in a subject, comprising:
(a) obtaining a sample from said subject;
(b) determining the level of C-reactive protein (CRP) in said sample; and
(c) administering granulated mesalamine to the subject if the determined level of CRP is at least about 2.2 mg/L,
wherein administration of granulated mesalamine results in the improvement of symptoms of the gastrointestinal disorder.

12. A method of improving the symptoms of a gastrointestinal disorder in a subject, comprising:
(a) obtaining a sample from said subject;
(b) determining the level of C-reactive protein (CRP) in said sample; and
(c) administering granulated mesalamine to the subject if the determined level of CRP is at least about 4 mg/L,
wherein administration of granulated mesalamine results in the improvement of symptoms of the gastrointestinal disorder.

13. The method of any one of claims 6-12, wherein the sample is a blood sample.

14. The method of any one of claims 6-12, wherein the level of CRP is determined by a high-sensitivity CRP (hs-CRP) test.

15. The method of any one of claim 1-3 or 6-12, wherein the gastrointestinal disorder is selected from the group of: an inflammatory gastrointestinal disorder, irritable bowel disease, a gastrointestinal motility disorder, a functional gastrointestinal disorder, gastroesophageal reflux disease (GERD), Crohn's disease, ulcerative colitis, diverticulitis, inflammatory bowel disease, and gastroparesis.

16. The method of any one of claim 1-3 or 6-12, wherein the subject is administered between about 0.75 grams and 3 grams of mesalamine per day.

17. The method of any one of claim 1-3 or 6-12, wherein the mesalamine is administered as a multiple of a unit dosage of 375 mg.

18. The method of any one of claim 1-3 or 6-12, wherein the mesalamine is provided as a delayed and/or extended release formulation.

19. The method of any one of claim 1-3 or 6-12, wherein the mesalamine is provided as an oral formulation.

20. The method of any one of claim 1-3 or 6-12, wherein the mesalamine is provided as a solid dosage unit.

21. The method of claim 20, wherein the solid dosage unit is a tablet or capsule.

22. The method of claim 20, wherein the solid dosage unit is coated with a pH dependent enteric coating.

23. The method of claim 22, wherein the coating dissolves at a pH of between about 5.5 and 7.

24. The method of any one of claims 9-12, wherein administration of the mesalamine results in the improvement of at least one of the following: abdominal pain, stool consistency, daily symptoms, and bloating.

25. The method of claim 15, wherein the wherein the gastrointestinal disorder is diarrhea-predominant IBS (d-IBS).

26. A method of treating a gastrointestinal disorder in a subject, comprising administering between about 0.75 grams and 3 grams of granulated mesalamine to the subject, wherein administration of the granulated mesalamine results in treatment of the gastrointestinal disorder.

27. The method of claim 26, wherein the gastrointestinal disorder is selected from the group of: an inflammatory gastrointestinal disorder, irritable bowel disease, a gastrointestinal motility disorder, a functional gastrointestinal disorder, gastroesophageal reflux disease (GERD), Crohn's disease, ulcerative colitis, diverticulitis, inflammatory bowel disease, and gastroparesis.

28. The method of claim 26, wherein the mesalamine is administered as a multiple of a unit dosage of 375 mg.

29. The method of claim 26, wherein the mesalamine is provided as a delayed and/or extended release formulation.

30. The method of claim 26, wherein the mesalamine is provided as an oral formulation.

31. The method of claim 26, wherein the mesalamine is provided as a solid dosage unit.

32. The method of claim 31, wherein the solid dosage unit is a tablet or capsule.

33. The method of claim 31, wherein the solid dosage unit is coated with a pH dependent enteric coating.

34. The method of claim 33, wherein the coating dissolves at a pH of between about 5.5 and 7.

35. The method of claim 26, wherein administration of the mesalamine results in the improvement of at least one of the following: abdominal pain, stool consistency, daily symptoms, and bloating.

36. The method of claim 27, wherein the wherein the gastrointestinal disorder is diarrhea-predominant IBS (d-IBS).

37. The method of claim 3, further comprising selecting the subject for treatment based on the level of CRP in said subject.
